(19) United States
(12) Patent Application Publication
(10) Pub. No.: US 2025/0251703 A1
(43) Pub. Date: Aug. 7, 2025

(54) INPUT DEVICE WITH CONDUCTIVE SIGNAL PATH USING CONDUCTIVE VISCOUS MATERIAL

(71) Applicant: Apple Inc., Cupertino, CA (US)

(72) Inventors: Antonio F. Herrera, Woodside, CA (US); Junlin Mu, Santa Clara, CA (US)

(21) Appl. No.: 18/431,868

(22) Filed: Feb. 2, 2024

(51) Int. Cl. G04G 21/02; A61B 5/00; A61B 5/024; A61B 5/33; G04G 21/08

(52) U.S. Cl. CPC G04G 21/025; A61B 5/33; A61B 5/681; A61B 5/7455; G04G 21/08; A61B 5/02438; A61B 5/02444

(57) ABSTRACT

A crown assembly of an electronic device may be configured to receive translational inputs, rotational inputs, and electrocardiogram (ECG) signals via a user input surface of a knob of the crown. The crown may include a shaft that extends from the knob at a first end and terminates at a second end, opposite the first end. The second end of the shaft may be conductively coupled to a conductive member of the crown assembly. A conductive viscous material may be positioned at an interface between the second end of the shaft and the conductive member. The shaft, the conductive viscous material, and the conductive member may define an electrical path carrying the ECG signal. A reservoir positioned at the interface area is defined by either the second end of the shaft, the conductive member, or both, and is filled with the conductive viscous material to help maintain the electrical path.

### TECHNICAL FIELD

The subject matter of this disclosure relates generally to electronic devices, and more particularly, to electronic devices with user input assemblies that include a reservoir for a conductive viscous material configured to electrically conduct signals from the user input assembly.

### BACKGROUND

Electronic devices such as mobile phones, tablet computers, wearable devices, and the like may include input systems, such as buttons, crowns, dials, and the like, which can detect a variety of inputs or other signals. For example, a watch or other electronic device may include a button that can be pushed in order to provide inputs to the device. As another example, a watch may include a crown that can receive rotational and/or translational inputs. Such input systems may be used in various combinations to provide input functionality to a device.

### SUMMARY

An electronic watch described herein may include a housing having a sidewall, the sidewall defining an opening; a crown defining a user input surface and extending through the opening, the crown configured to receive a least one of a rotational input or a translational input. The crown may include a knob defining the user input surface and a shaft extending from the knob. The shaft may define a first end coupled to the knob and a second end opposite the first end, the second end defining a reservoir. The electronic watch may include a sensing system configured to receive an electrocardiogram (ECG) signal from the user input surface, a dome switch configured to produce haptic feedback in response to the translational input at the user input surface, a conductive member conductively coupling the shaft to the sensing system, the conductive member positioned between the dome switch and the second end of the shaft, and a conductive viscous material between the second end of the shaft and the conductive member, the conductive viscous material filling the reservoir and configured to conductively couple the user input surface to the conductive member.

In some examples, the shaft defines a central axis around which the crown rotates and the reservoir is centered about the central axis. In some cases, the reservoir is a first reservoir. The first reservoir may be defined by a curved surface depressed with respect to the second end. The conductive member may be defined a second reservoir positioned opposite the first reservoir and the conductive viscous material may fill the second reservoir. In some examples, conductive member comprises a stiffening plate at least partially surrounding the second reservoir, the stiffening plate configured to contact the dome switch in response to the translational input.

In some embodiments, conductive member is configured to apply a biasing force to the first end of the shaft, the biasing force biasing the crown towards an undepressed position. In some cases, the shaft defines a bottom surface of the reservoir and a wall surface of the reservoir. The conductive member may be positioned at least partially in the reservoir and the conductive viscous material may be between the bottom surface of the reservoir and the conductive member. In some examples, the portion of the conductive member contacts the wall surface of the reservoir, thereby defining a seal between the portion of the conductive member and the peripheral wall.

According to some examples described herein, an electronic watch includes: a processor; a housing including a sidewall, the sidewall defining an opening; a front cover coupled to the housing; a display positioned below the front cover; a flexible circuit element within the housing; a dome switch positioned within the housing; a crown, a conductive member, and a conductive viscous material. The crown may be positioned along the sidewall of the housing and defining a user input surface configured to receive a translational input. The crown may include a shaft configured to actuate the dome switch, the shaft extending through the opening and defining an interface surface opposite the user input surface, the interface surface defining a reservoir. The conductive member may be configured to conduct an electrical signal from the user input surface to the processor, the conductive member positioned between the dome switch and the crown and conductively coupling the crown to the flexible circuit element. The conductive viscous material may be positioned between the conductive member and the interface surface of the shaft, the conductive viscous material filling the reservoir and conductively coupling the shaft to the conductive member.

In some examples, the shaft includes a shaft member having a first end and a second end opposite the first end, the first end of the shaft member coupled to the user input surface of the crown and a barrel coupled to the second end of the shaft member, the barrel defining the interface surface and the reservoir. The crown may be configured to rotate with respect to a central axis of the shaft in response to a rotational input from a user at the user input surface. In some cases, the conductive viscous material is an electrically conductive grease.

In some embodiments, the interface surface of the crown is configured to, in response to a rotational input applied to the crown, rotate with respect to the conductive member, thereby applying a shear force to the conductive viscous material. In some examples, the crown, the conductive viscous material, the conductive member, and the flexible circuit element define a conductive path from the user input surface to the flexible circuit element, and the electrical signal is a physiological signal from a user's skin.

In some examples, the conductive member is in contact with the interface surface, the conductive member is configured to deform in response to the translational input, and the conductive viscous material is configured to flow during the deformation of the conductive member. the reservoir is a reservoir of a plurality of reservoirs having a concentric pattern along the interface surface.

A wearable device described herein may include: a housing having a sidewall, the sidewall defining an opening; a sensing system configured to receive an electrocardiogram (ECG) signal; a crown assembly; a conductive member; and a conductive viscous material. The crown may define a user input surface configured to receive the ECG signal, the crown may extend through the opening and include a shaft. The shaft may define a first end portion conductively coupled to the user input surface and a second end portion opposite the first end portion. The conductive member may be configured to apply a biasing force against the second end portion of the shaft. A conductive viscous material may be between the conductive member and the second end portion of the shaft, the conductive viscous material may be configured to conductively couple the conductive member and the shaft, the conductive viscous material conforming to the second end portion of the shaft. The crown assembly may have a reservoir defined by at least one of the conductive member or the second end portion of the shaft and the conductive viscous material may fill the reservoir.

The shaft may include a shaft member defining the first end portion of the shaft and extending from the housing; and a barrel coupled to the shaft member and defining the second end portion of the shaft, the barrel defining an interface surface configured to contact the conductive viscous material. The barrel may define a peripheral wall that defines the reservoir; and the conductive member may be positioned partially within the peripheral wall trapping the conductive viscous material. In some examples, the conductive member may define the reservoir; the shaft further includes a barrel defining the second end portion of the shaft and configured to contact the conductive viscous material; and the end portion of the barrel is positioned within the reservoir, thereby trapping the conductive viscous material between the reservoir and the end portion of the barrel.

In some examples, the reservoir is a first reservoir, the first reservoir is defined by the second end portion of the shaft, the crown assembly further includes a second reservoir defined by the conductive member; and the first and second reservoirs are centered with respect to an axis of rotation of the input member. In some cases, the wearable device further includes a sealing member and the sealing member is concentric to the reservoir, thereby sealing a portion of the conductive viscous material with respect to the second end portion of the shaft and the conductive member.

In some examples, the reservoir is centered with respect to a rotation axis of the second end portion of the shaft, the second end portion defines an interface surface contacting the conductive viscous material, and the reservoir is defined by a reservoir wall extending orthogonally from the interface surface in a direction opposite the interface surface.

### BRIEF DESCRIPTION OF THE DRAWINGS

Reference will now be made to representative embodiments illustrated in the accompanying figures. It should be understood that the following descriptions are not intended to limit this disclosure to one included embodiment. To the contrary, the disclosure provided herein is intended to cover alternatives, modifications, and equivalents as may be included within the spirit and scope of the described embodiments, and as defined by the appended claims.

FIGS. 1A and 1B depict an example electronic device including a crown assembly with a reservoir, such as described herein.

FIG. 2 depicts a cross-sectional view of an example crown assembly with a reservoir, such as described herein.

FIGS. 3A-3C depict detailed views of example reservoir configurations, such as described herein.

FIGS. 4A-4B depict detailed views of example reservoir configurations, such as described herein.

FIG. 5 depicts a detailed view of an example reservoir configuration, such as described herein.

FIG. 6 depicts a detailed view of an example reservoir configuration, such as described herein.

FIGS. 7A-7B depicts a detailed view of an example reservoir configuration, such as described herein.

FIG. 8 depicts example components of the electronic device.

The use of the same or similar reference numerals in different figures indicates similar, related, or identical items.

The use of cross-hatching or shading in the accompanying figures is generally provided to clarify the boundaries between adjacent elements and also to facilitate legibility of the figures. Accordingly, neither the presence nor the absence of cross-hatching or shading conveys or indicates any preference or requirement for particular materials, material properties, element proportions, element dimensions, commonalities of similarly illustrated elements, or any other characteristic, attribute, or property for any element illustrated in the accompanying figures.

Additionally, it should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Embodiments described herein relate to electronic devices. In particular, embodiments described herein are directed to a crown assembly that includes a reservoir at an interface between two members that move with respect to each other in response to user rotational and translational inputs. The reservoir traps a conductive viscous material therein to maintain electrical continuity between the two members throughout repeating dynamic cycles.

Small electronic devices, such as smart watches and other wearable devices, often include user input controls that can receive a variety of inputs. For example, a smart watch may include a crown assembly that can receive rotational and translational inputs, as well as electrical signals like an electrocardiogram (ECG) signal from a user. In this example, the smart watch can include an ECG sensor that detects a user's heart rate when the user contacts an external input surface (e.g., an electrode) of the crown assembly. The ECG signal may travel through portions of the crown before reaching the ECG sensor. To achieve greater accuracy when measuring an ECG signal via an electrode, a low-electrical resistance electrical path for the ECG signal can reduce attenuation of the signal or otherwise provide a better signal transmission performance. However, over time and with continuous rotational and translational inputs, the electrical connection between members along the electrical path may degrade or erode (e.g., due to shear and pressure between the members, and/or other phenomena). This degradation may increase the electrical resistance between the members, resulting in intermittent conductivity or no conductivity, or otherwise negatively affecting the ECG signal. Such effects may result in poor or no ECG signal reaching the ECG sensor, may decrease a signal-to-noise ratio, or otherwise negatively effect the operation of the ECG sensor.

In some examples, a conductive viscous material, such as a conductive grease or other lubricant, may be positioned between the members that have non-fixed (e.g., subject to dynamic loading and/or frictional interfaces) couplings. However, due to its physical properties (e.g., viscous), the conductive viscous material may flow or shift away from the contact interface between the members that are under the dynamic loading (e.g., due to frictional forces). For example, the interface may be an area where the members are in contact with each other and where one member may rotate with respect to the other member (e.g., the end of a rotating shaft may contact and rotate against a friction guard or other non-rotating component). In this example, the forces resulting from the relative movement of the components may cause the conductive viscous material to shift away from the interface, resulting in depleting the conductive viscous material at the interface and thus increased friction and decreased electrical conductivity between the members.

A crown assembly, such as described herein, may include a reservoir at an interface between two members that are in physical and electrical contact. A conductive viscous material may be positioned at this interface (e.g., between the two members) and may fill the reservoir. In some examples, at least one of the members defines the reservoir and a portion of conductive viscous material that fills the reservoir is maintained within the reservoir over time and/or throughout repeated dynamic cycles, thereby maintaining the electrical connectivity between the two members. The reservoir traps and/or captures the conductive viscous material therein, such that a conductive connection between the members is maintained even through repeated movement of the members relative to one another (e.g., through the conductive viscous material).

In some embodiments described herein, the crown assembly may include an external portion (e.g., a knob) that defines an external surface of a crown. A shaft may extend from the knob (e.g., from a first end of the shaft) and through the housing. A second end of the shaft (e.g., opposite the first end) may contact a conductive member of the crown assembly. The second end of the shaft may be configured to be conductively coupled to the conductive member. The second end of the shaft may also transfer a translational input applied by a user at the knob to a dome switch of the crown assembly. In some cases, the conductive member acts as a friction guard between the shaft and the dome switch, such that friction from shaft rotation is applied to the conductive member instead of the dome switch. The shaft may rotate in response to a rotational input received at the knob, and an optical detector of the crown assembly may detect the rotation of the shaft. While the shaft and the conductive member are in contact with each other (e.g., not fixed), the shaft is configured to be free to rotate with respect to the conductive member while the conductive member remains fixed (e.g., the conductive member does not rotate). In addition, the conductive member is configured to deflect in response to the translational input at the knob, thereby maintaining contact with the shaft. The reservoir may be defined by the second end of the shaft, the conductive member, or both. A conductive viscous material is between the second end of the shaft and the conductive member and fills this reservoir.

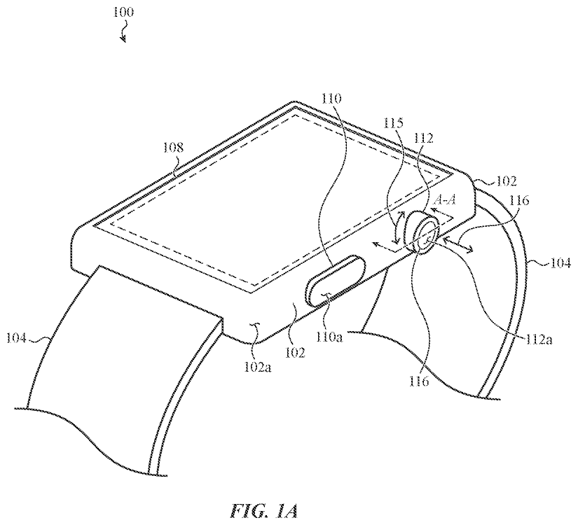

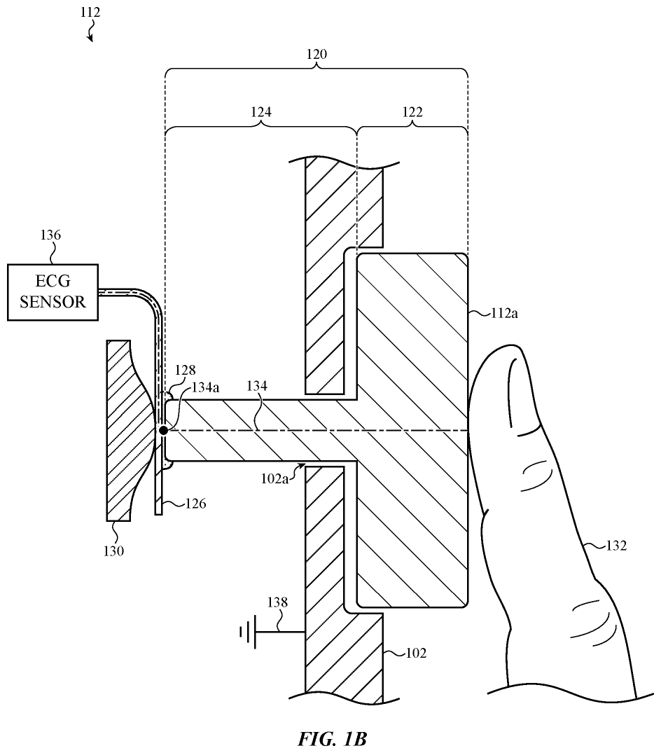

These foregoing and other embodiments are discussed below with reference to FIGS. 1A-8. However, those skilled in the art will readily appreciate that the detailed description given herein with respect to these figures is for explanation only and should not be construed as limiting.

FIG. 1A depicts a perspective view of an example electronic device 100 (or device 100) which may include an input member with a reservoir at an interface between two members in conductive contact with each other, such as described herein. In some cases, the device 100 is a watch. In other cases, the user input control may be incorporated into electronic devices including phones, tablets, laptops, AR/VR headsets (e.g., head mounted displays), biometric sensing and/or health monitoring devices, headphones, digital media players, and the like.

The device 100 may include a housing 102. The housing 102 may be configured to house internal components of the device, including batteries, processors, sensors, and the like. In some cases, the housing is formed from a metal, such as titanium, aluminum, steel, or any alloys or combination of materials, as may be known to one of skill in the art. In some examples, the housing is formed from ceramic, glass, polymers, and the like. The housing 102 may be a monolithic piece or may be assembled from multiple parts using any suitable method. The housing may include openings and/or recesses that receive and/or secure user input controls and/or electronic components to the device 100.

A display 108 may be positioned within the housing 102. A cover may be positioned over the display. The cover may be glass, sapphire, polymer, or any suitable material, as may be known to one of skill in the art. More generally, the display may be a liquid crystal display (LCD), an organic light emitting diode display (OLED), or any suitable display technology. The display 108 outputs a graphical user interface of the device and may include other sensors, such as a fingerprint sensor, touch sensors, force sensors, and the like. The touch and/or force sensors may detect various types of user interactions including swipes, taps, and other gestures.

The device 100 may also include a wristband 104 that secures the device 100 to a user's wrist. In some cases, the wristband 104 is detachably coupled to a recess or other feature of the housing 102. In some cases, the coupling portion of the wristband 104 may be formed from the same material as the housing 102. In other examples, the wristband 104 is formed from other materials to provide a visually distinct appearance.

The housing 102 may define an external side surface 102a along which various user input controls may be positioned. For example, the device 100 includes a side button 110 and a crown assembly 112 positioned along (and which may partially protrude from) the external side surface 102a. The button 110 may be configured to be translated inward in response to a user input (e.g., via input surface 110a), and may be programable such that the user can define a particular function that is controlled by the button.

The crown assembly 112 may protrude from the external side surface 102a of the housing 102. In some examples, at least a portion of the crown assembly 112 is external to the housing 102. More specifically, a knob of the crown assembly 112 may be external to the housing 102. The knob of the crown assembly 112 may define a generally round shape with texture, knurling, grooves, or other patterns that facilitate gripping of the crown assembly (e.g., during rotation of the crown). In some cases, the crown assembly 112 is configured to receive a variety of inputs through which users can interact. For example, a user may rotate the crown assembly 112 (interaction 115) and/or may translate the crown assembly 112 (interaction 116) to zoom, scroll, rotate, select, or otherwise change the user interface of the display 108. In some cases, rotation of the crown assembly 112 may be detected via self-mixing interferometry or other suitable methods as may be known to one of skill in the art.

In some examples, the crown assembly 112 can receive biometric signals from a user. For example, the crown assembly may be coupled to biometric sensing circuitry or include or define conductive paths to biometric circuitry within the device 100. More particularly, the knob of the crown assembly 112 may define a user input surface 112a through which electrical signals, such as physiological signals can be received. For example, the device 100 may receive biometric signals, heart rate and/or ECG signals, galvanic skin response signals, and the like when a user's skin contacts the crown assembly 112 at the user input surface 112a. Specifically, the sensors may receive the ECG signals when the user opens an application or otherwise activates the function, in some examples. More generally, the user input surface 112a may be a conductive surface (e.g., formed from a metal or other conductive material) that is coupled to biometric sensors. When used to conductively couple to a user's skin for detecting signals (e.g., voltage signals), the user input surface 112a may be referred to as an electrode.

FIG. 1B shows a simplified cross-sectional view along line A-A of FIG. 1A. As depicted, a crown assembly 112 may generally include a crown 120 which, in turn, may include a knob 122 and a shaft 124. The crown assembly 112 may also include a conductive member 126, a conductive viscous material 128, and a dome switch 130.

At the crown 120, the knob 122 may define the user input surface 112a of the crown assembly 112. The user input surface 112a may be or may act as an electrode configured to receive a signal from a user's skin or user's finger 132. The shaft 124 may be coupled to the knob 122 at a first end of the shaft and extend from the knob 122 through an opening of the housing 102 (e.g., defined by a sidewall of the housing). The shaft 124 may define a second end, opposite the first end, that defines an interface surface. This interface surface conductively and dynamically interfaces with the conductive member 126 and with the conductive viscous material 128. More specifically, a conductive viscous material 128 is positioned between the second end of the shaft 124 and the conductive member 126 to provide a conductive connection between the shaft 124 and the conductive member 126. The conductive member 126 contacts the shaft 124 (e.g., at the second end) and may apply a biasing force to maintain this contact (and optionally to help bias the crown 120 in an undepressed position).

Due to the configuration described above, an electrical path 134 is defined between the user input surface 112a and an ECG sensor 136, which can carry or transmit an ECG signal from a user's finger when the finger is in contact with the user input surface 112a. The ECG signal travels via electrical path 134, which includes a portion of the knob 122 that defines the user input surface 112a, the shaft 124, the conductive viscous material 128, and the conductive member 126. The conductive member 126 may couple to the ECG sensor via a flexible board or the like. In some cases, at node 134a, the contact between the shaft 124 and the conductive member 126, without the conductive viscous material 128, may provide a conductive contact to define the electrical path 134. However, due to the dynamic loads between the shaft 124 and the conductive member 126, the conductive viscous material 128 provides an additional interface or conductive pathway that reduces the electrical resistance between the shaft 124 and the conductive member 126 to improve the signal quality that is received at the ECG sensor 136. For example, the conductive viscous material 128 may ensure a sufficient conductive coupling even when the shaft 124 is rotated or dynamically pressed against the conductive member 126.

As depicted in FIG. 1B, the electrical path 134 extends between and/or conductively couples the electrode at user input surface 112a and the ECG sensor 136. Accordingly, while both the crown 120 and the housing 102 may be formed from or include conductive materials, the crown 120 is electrically isolated from the housing 102 to prevent creating a ground path 138 between the crown 120 and the housing 102 that may interfere with the signal to the ECG sensor 136.

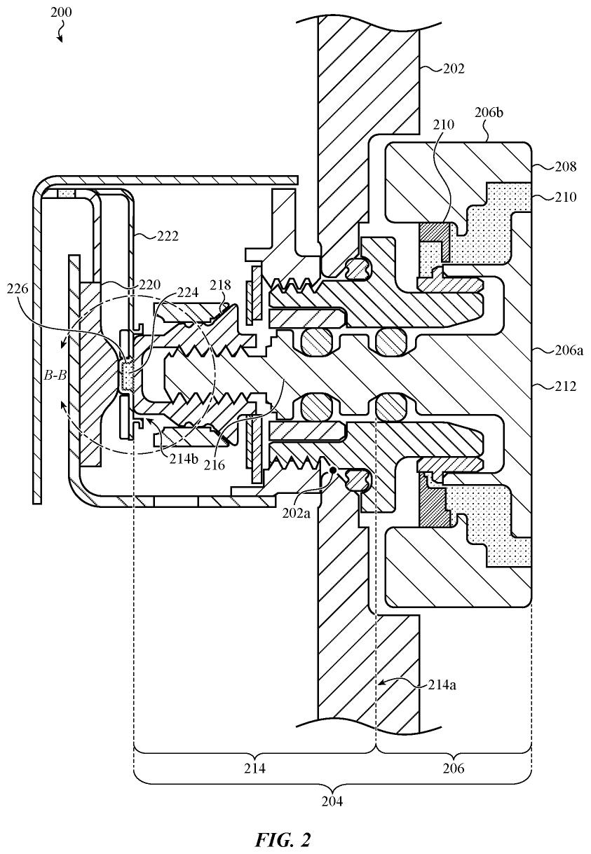

FIG. 2 shows a cross-sectional view of a crown assembly 200 along line A-A of FIG. 1A. As described herein, the crown assembly 200 generally refers to the crown, switches, and structural and electrical components that may be used to operate the crown. In particular, a crown assembly 200 may include a crown 204 which can extend through a portion of a sidewall 202 of a housing of an electronic device.

The crown 204 may protrude from the sidewall, allowing a user to press the crown (e.g., translational input), rotate the crown (e.g., rotational input), touch the crown (e.g., touch and/or ECG input), and the like, to obtain different outputs displayed on a display of the device and/or to control other device functions. In particular, the crown 204 may include a knob 206 which defines the portion of the crown 204 that the user interacts with. For example, the knob 206 may define a user input surface 206a. The user input surface 206a may be configured to receive a translational (or axial) input from a user (e.g., by a user's finger pressing the crown inwards) and may be configured to receive an ECG signal from a user, as depicted in FIG. 1B. The knob 206 may also include a side user input surface 206b which may be configured to receive a rotational input from a user. In some examples, the knob 206 may additionally include a ring portion 208 which defines the side user input surface 206b and which may surround a medial portion 212 of the knob 206. In some cases, the ring portion 208 and the medial portion 212 may be electrically isolated from each other by an isolating portion 210. In some cases, the isolating portion 210 may include one or more molded components and/or isolating gaskets positioned along the knob 206 to isolate the ring portion 208 from the medial portion 212. In some configurations, the knob 206 is a monolithic piece formed from a metal material, in which the monolithic piece defines the user input surface 206a and the side user input surface 206b.

A shaft 214 of the crown 204 may extend from the knob 206. The shaft 214 is generally configured to transfer translational (and/or axial) inputs received at the user input surface 206a to a dome switch 220 (e.g., actuate the dome switch by pressing an end of the shaft 214 against the dome switch 220), or to another force sensing element or system. The shaft 214 may also be used to facilitate rotation sensing, as the shaft rotation may be detected by an optical encoder or other rotational sensing system(s).

The shaft 214 may define a first end 214a and a second end 214b (opposite the first end 214a). The first end 214a may be coupled to the knob 206 and/or may be coupled to the user input surface 206a. In some embodiments, the shaft 214 and the knob 206 may be a monolithic piece formed from a conductive material, such as steel, titanium, aluminum, or other materials or combination of materials.

In some embodiments, the shaft 214 may include a shaft member 216 and a barrel 218. The shaft member 216 may be an elongate piece that defines the main body of the shaft 214 and which extends through an opening 202a of the sidewall. The shaft member 216 may be conductively coupled to the user input surface 206a, thereby enabling an ECG signal to travel through the shaft 214. The barrel 218 may be fixedly coupled to an end portion of the shaft member 216 and may include one or more components. The barrel 218 may be conductively coupled to the shaft member 216 and may convey the ECG signal from the shaft member 216 to the conductive member 222.

In some cases, the barrel 218 may be an optical barrel that defines a surface that reflects light for an optical sensor to user for detecting rotation. The optical sensor may include an emitter that emits light onto the surface of the barrel 218 and a receiving that detects incident light from the surface to measure and/or detect a rotation of the crown 204 (e.g., applied via the knob 206). In some examples, the barrel 218 is configured to rotate with the shaft member 216 in response to rotational inputs and translate with the shaft member 216 in response to translational inputs. In some cases, the barrel 218 defines the second end of the shaft 214, which interfaces with the dome switch 220 and a conductive member 222. For example, the conductive member 222 and/or the dome switch 220 may push against an end portion of the barrel to maintain the crown in an undepressed position. In some examples, the barrel 218 and the shaft member 216 may be a monolithic piece.

As explained above, the crown assembly 200 may include a conductive member 222. As depicted in FIG. 2, a portion of the conductive member 222 is between the shaft 214 (e.g., at second end 214b) and the dome switch 220. In some examples, the conductive member 222 is configured to conduct the ECG signal (or other electrical signals) from the user input surface 206a to a sensing system (e.g., ECG sensing system shown in FIG. 1B) or other processors within the watch. In some configurations, the conductive member 222 is configured to apply a biasing force against the shaft 214 (via the barrel 218, if the assembly includes the barrel). The biasing force may be applied to bias the crown in the undepressed positioned. In some cases, the biasing force maintains contact between the second end 214b of the shaft 214 and the conductive member 222. By maintaining contact, electrical continuity between the shaft 214 and the conductive member 222 is achieved. As discussed as to FIG. 1B, the electrical contact between the shaft 214 and the conductive member 222 conveys the ECG signal to the sensing system.

Due to the configuration of the conductive member 222 applying a biasing force against the shaft 214, the conductive member 222 is configured to deform, translate, or otherwise move in response to a translational input at the user input surface 206b. Thus, when the input surface 206b is pressed, the conductive member 222 moves with the crown to a depressed position and actuates (e.g., compresses) the dome switch (or other force sensing element). For example, the conductive member 222 may move a same distance as the crown and may react against the dome switch 220 to compress it. Upon release of the crown (e.g., to the undepressed position), the conductive member 222 springs back with the crown 204 while maintaining contact with the end 214b of the shaft 214. Similarly, when the crown 204 is caused to rotate (e.g., due to a rotational input), the end 214b of the shaft 214 may rotate against the conductive member 222. The conductive member 222 may be static while the end 214b of the shaft 214 moves relative to it. Due to this structure, in some examples, the conductive member 222 may be movably (e.g., rotatably and translatably) coupled to the shaft 214.

As explained above, the conductive member 222 may be-directly and/or indirectly (via a thin film of conductive viscous material)-in contact with the end 214b of the shaft. Further, the conductive member 222 and the end 214b of the shaft may be under dynamic loading and/or frictional forces (e.g., due to the rotation and translation cycles over time). In some embodiments, the crown assembly 200 includes a conductive viscous material 224 which conductively couples the conductive member 222 to the shaft 214. For example, the conductive viscous material 224 may provide a thin buffer between conductive member 222 and the shaft 214. The conductive viscous material 224 may have low electrical resistivity and thus may be configured to conduct electricity from the shaft 214 to the conductive member 222. In some embodiments, the conductive viscous material 224 is configured to increase the electrical contact surface area between the shaft 214 and the conductive member 222, thereby improving the electrical connection between these members. The conductive viscous material 224 may also be configured as a lubricant to reduce the friction between the conductive member 222 and the end 214b. The conductive viscous material 224 may be lubricants, such as gels, oils, greases, semisolid materials, liquids, and the like. In some examples, the conductive viscous material 224 may be a carbon-based or a silicone-based grease with high electrical conductivity.

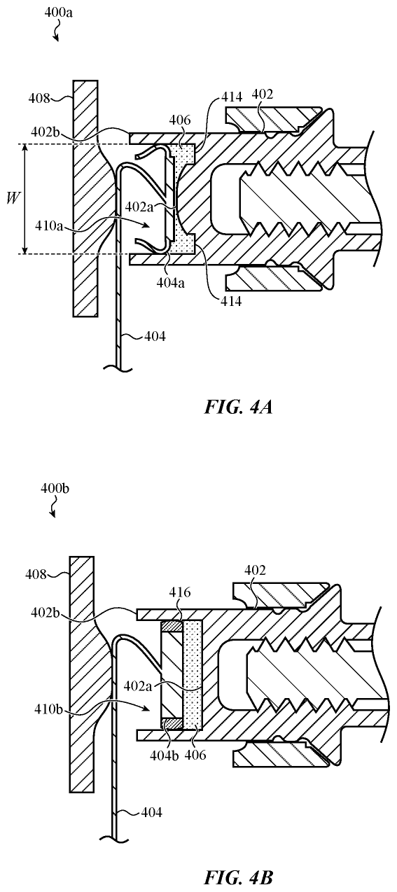

Due to the biasing force, the shear force (e.g., due to rotation), and frictional forces applied by the shaft 214 on the conductive member 222, the conductive viscous material 224 may, over time, flow away from this interface area (e.g., due to compression, shear forces, and/or other phenomena). Depletion or displacement of the conductive viscous material can diminish the quality of the ECG signal (e.g., increasing electrical resistance of the conductive path, decreasing signal to noise ratio, etc.). Accordingly, a reservoir 226 may be positioned at the interface area to maintain at least a portion of the conductive viscous material 224 therewith. For example, the reservoir may be configured to reduce the dynamic loading, compression forces, shear forces, etc., experienced by the portion of the conductive viscous material 224 filling the reservoir. As depicted in FIG. 2, the reservoir 226 may be defined by the conductive member 222. In some embodiments, the reservoir 226 may be defined by the end 214b of the shaft. Details and configurations of the reservoir 226 are described in FIGS. 4A-7 below. In general, the reservoir 226 traps a portion of the conductive viscous material 224 such that, even if depletion and/or displacement occurs in the frictional interfaces, the trapped portion maintains electrical continuity between the members. In some embodiments, described below, the reservoir may be centered with respect to a central axis of the shaft (e.g., an axis about which the shaft rotates) to reduce the magnitude of rotational loading applied at this interface, compared to peripheral areas of the shaft end.

In some cases, the conductive member 222 may be configured to measure a force applied by the user to the crown. For example, the conductive member 222 may include two conductive members and a compliant member between the conductive members. This sandwich may be configured to detect a change in capacitance. In some examples, the conductive member 222 may include a strain sensing system or other force measurements sensors that detect a force applied to the crown. In some cases, different magnitudes of force may trigger different graphical outputs on the display.

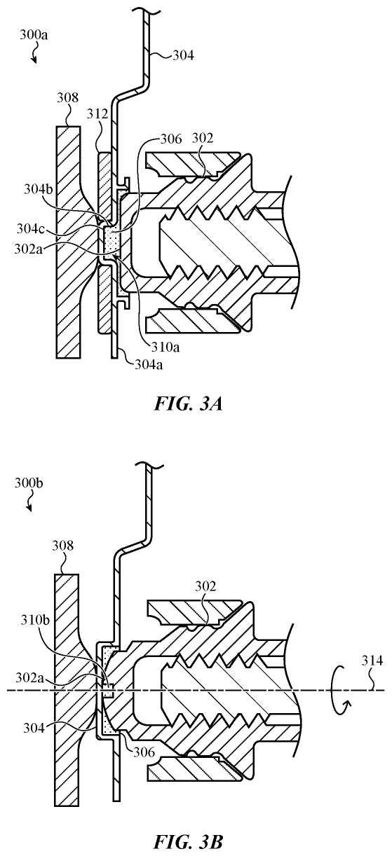

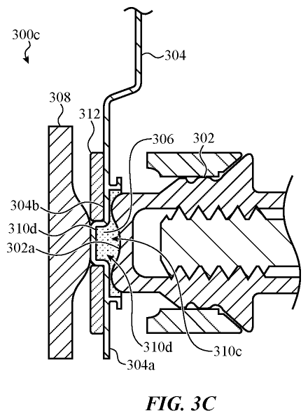

FIG. 3A shows a detail view of an example reservoir configuration 300a, as discussed in FIG. 2. As depicted, an end portion of a shaft 302 may include an interface surface 302a which is in contact with a conductive member 304 and with a conductive viscous material 306 between the end portion of the shaft 302 and the conductive member 304. The conductive member 304 may define a reservoir 310a which is positioned opposite the interface surface 302a. The reservoir 310a may be a depression with respect to a surface 304a of the conductive member that is in contact with the conductive viscous material 306. In some examples, a wall 304b of the reservoir extends perpendicularly with respect to surface 304a. A bottom of the reservoir 304c may be perpendicular with respect to surface of the wall 304b. In some examples, however, the reservoir 310a may be curved or have another other suitable shape for containing the conductive viscous material 306 therein and/or for reducing frictional interfaces for a portion of the conductive viscous material 306. In some examples, surface 304a and interface surface 302a are configured to be in contact while maintaining a clearance with respect to the bottom of the reservoir 304c. In this configuration, at least a portion of the conductive viscous material is not subject to the dynamic loading from the rotational and translational input. Stated another way, the conductive viscous material 306 that is in the reservoir 310a may be less affected by the shear and compression forces applied by the interface surface 302a during rotational and translational inputs, and thus may tend to remain in the reservoir during such inputs. Accordingly, the portion of the conductive viscous material (within the reservoir) that is not dynamically loaded (or experiences less dynamic loading or better tolerates the dynamic loading) stays in the reservoir and therefore can maintain electrical continuity between the shaft 302 and the conductive member 304, even if the conductive viscous material directly between the interfacing surfaces has depleted (e.g., by shifting away from the interface). In some examples, as depicted in FIG. 3A, the reservoir 310a may be smaller (e.g., have a smaller diameter) than the end of the shaft 302, such that the reservoir 310a is completely covered by the end of the shaft 302. Stated another way, the reservoir 310a may be sized, relative to the end of the shaft, such that the end of the shaft creates a seal effect around the reservoir.

In some examples, the conductive member 304 may include a stiffener 312 which may surround the reservoir 310a. The stiffener 312 may be configured to stiffen a portion of the conductive member 304 that presses on the dome switch, and/or to provide a flat surface (or to generally allow customization of the surface that contacts the dome switch). For example, the stiffener 312 may reduce warping of the conductive member 304 during a translational input.

FIG. 3B shows a variation of an example reservoir configuration 300b. In this variation, a reservoir 310b is defined by the shaft 302 instead of by the conductive member 304. In this configuration, the conductive member 304 may be easier to manufacture and provide a more uniform surface for the conductive surface to react against the dome switch 308 without additional stiffeners. Similar to FIG. 3A, at the bottom of reservoir 310a, a portion of the conductive viscous material 306 may not be under dynamic loading and/or frictional interface and thus is less likely to deplete or be displaced due to rotational and axial inputs, thereby maintaining electrical continuity between the shaft 302 and the conductive member 304. Put differently, a portion of the conductive viscous material 306 in the reservoir 310b may be less affected by the shear and compression forces applied by the interface surface 302a during rotational and translational inputs, and thus may tend to remain in the reservoir 310b during such inputs. In some examples, the reservoir 310b may be centered with respect to a rotational axis 314 of the shaft. In this example, the magnitude of the shear forces applied at the reservoir 310b area are reduced because the reservoir is closer and/or centered with respect to the rotational axis of the shaft 302.

FIG. 3C shows a variation of an example reservoir configuration 300c. In this variation, both the shaft 302 and the conductive member 304 include a reservoir 310c and 310d, respectively. Reservoir 310c may be defined by the shaft 302 and may have a curved surface that is depressed with respect to the interface surface 302a. Reservoir 310d may be defined by the conductive member 304. This reservoir 310d (which may have a similar configuration as the reservoir from FIG. 3A) may be opposite reservoir 310c, such that the two reservoirs cooperate to form a single reservoir volume. As depicted, reservoir 310d may define a curved surface 302a that helps seal the single reservoir volume. For example, the curved surface 302a defines peripheral points of contact between the shaft 302 and the conductive member 304, that traps the volume of the conductive viscous material 306 centrally and prevents it from shifting.

FIG. 4A shows a variation of an example reservoir configuration 400a. Unlike the variations of FIGS. 3A-3C, where the conductive viscous material fills the reservoir but may be otherwise free to flow, the reservoir configuration 400a traps the conductive viscous material between the shaft and the conductive member. In particular, a shaft 402 of the crown may define a reservoir 410a. The reservoir may be defined by a peripheral wall 402b that encircles or surrounds the conductive member 404. Due to this configuration, a portion 404a of the conductive member 404 is positioned within the reservoir 410a, thereby trapping the conductive viscous material 406. Since the portion 404a of the conductive member 404 is within the reservoir 410a, the interface surface 402a (e.g., a surface of the shaft that contacts the conductive viscous material 406 and the conductive member 404) may be defined by the bottom of the reservoir 410a. The portion 404a of the conductive member 404 inside the reservoir 410a (e.g., surrounded by peripheral wall 402b) may span a width W of the reservoir to provide a seal for the conductive viscous material 406. The portion 404a of the conductive member may be configured to be compressed against the inside of the peripheral wall 402b, thereby ensuring contact between the conductive member 404 and the shaft 402. For example, the portion 404a applies a biasing force against an inside surface of the peripheral wall 402b to define a metal-to-metal conductive path between the conductive member 404 and the shaft. This radial contact provides an additional path (in addition to the interface surface 402a and the conductive member 404 with the conductive viscous material in between) that conductively couple the conductive member 404 to the shaft. In some cases, the conductive member includes a surface for contacting the dome switch 408 to actuate it.

In some examples, the shaft 402 may define a subreservoir 414 within reservoir 410a. For example, the reservoir 410a may include a depression from interface surface 402a that reduces the dynamic loads and/or frictional interfaces compared to portions of the conductive member 404 that are in contact with surface 402a. This subreservoir 414 may behave like reservoirs 310a-d from FIGS. 3A-3B. Thus, if conductive viscous material 406 flows out of reservoir 410a, a portion of the conductive viscous material 406 still fills the subreservoir 414 to maintain electrical continuity.

FIG. 4B shows a variation of an example reservoir configuration 400b. In this variation, a portion 404b of the conductive member 404 that is surrounded by the peripheral wall 402b of the shaft 402 has a sealing member 416. The sealing member 416 may be configured to seal the conductive viscous material 406 within the reservoir 410b. The conductive sealing member 416 may be an overmolded compliant polymeric material, such as thermoplastic elastomers, liquid silicone rubber molding, or any combination of materials that is compatible with the conductive viscous material 406. In some cases, the sealing member 416 may electrically conduct electricity (e.g., an ECG signal). Due to the enhanced sealing between the portion 404b of the conductive material and the peripheral wall 402b, the interface surface 402a may be uniform to provide uniform pressure to the conductive viscous material 406 during dynamic load scenarios. In some examples, the interface surface 402a may define a dome shape or an otherwise at least partially curved shape to define a contact area between the shaft 402 and the conductive member 404 that conductively couples these members.

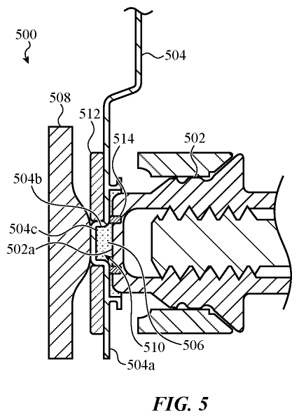

FIG. 5 shows an example reservoir configuration 500. In this variation, a sealing member 514 is positioned around a reservoir 510 to partially trap a conductive viscous material 506. More specifically, the reservoir configuration 500 may be similar to the configurations 300a-c from FIGS. 3A-3C. As depicted, a conductive member 504 may define the reservoir 510 and may be positioned centrally with respect to a rotation axis, in one example. The shaft 502 may include the sealing member 514, which may be a similar material from the sealing member 416. The conductive sealing member 514 may protrude from the interface surface 502a of the shaft. In some examples, the sealing member 514 is configured to contact and/or deform against contact surface 504a of the conductive member and may surround the reservoir 510. By surrounding the reservoir, an area of the conductive viscous material 506 is trapped between the reservoir 510 and the sealing member 514 to maintain electrical continuity between the shaft 502 and the conductive member 504. In this configuration, even if portions of the conductive viscous material 506 outside this area flows out (e.g., due to dynamic cycles and other frictional forces), a portion of the conductive viscous material 506 is maintained in the enclosed volume defined by the reservoir and the conductive seals to ensure electrical continuity of the shaft and the conductive member over time.

In some embodiments, an internal wall 504b of the reservoir 510 may extend perpendicularly from the surface 504a and perpendicularly from a bottom surface 504c of the reservoir. In some examples, other reservoir geometries are envisioned. The conductive member 504 may include stiffeners 512 that provide additional stiffness to a portion of the conductive member that reacts against and/or actuates the dome switch 508.

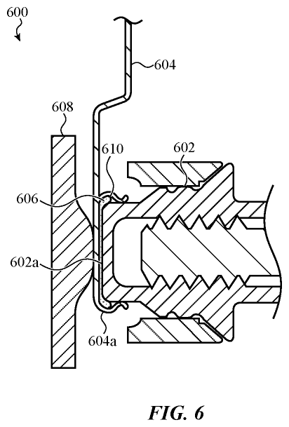

FIG. 6 shows a variation of an example reservoir configuration 600. In this variation, an end portion of a shaft 602 is positioned partially within a reservoir 610 defined by a conductive member 604. In some examples, the conductive member 604 may include a reservoir wall 604a which extends towards the shaft 602 (e.g., towards the right side of the page) to define the reservoir 610. The reservoir wall 604a may be configured to expand when the end of the shaft 602 is within the reservoir, thereby ensuring metal to metal contact (e.g., radially) between the shaft 602 (e.g., at the interface surface 602a) and the conductive member 604. While the reservoir wall 604a may apply a radially inward compressive force to the shaft 602 (e.g., to define a seal and maintain physical and electrical contact between the conductive member 604 and the shaft 602), the shaft is still configured to rotate in response to rotational inputs while the conductive member 604 remains static. The conductive viscous material 606 may be positioned between the conductive member 604 and the shaft 602. As depicted, the shaft 602 creates a partial seal with respect to the reservoir wall 604a (compare with FIG. 4A). As described above, a portion of the conductive member 604 defines a surface which is configured to actuate the dome switch 608 and which does not rotate with respect to the dome switch 608 (e.g., protects the dome switch against shear loads from the shaft 602).

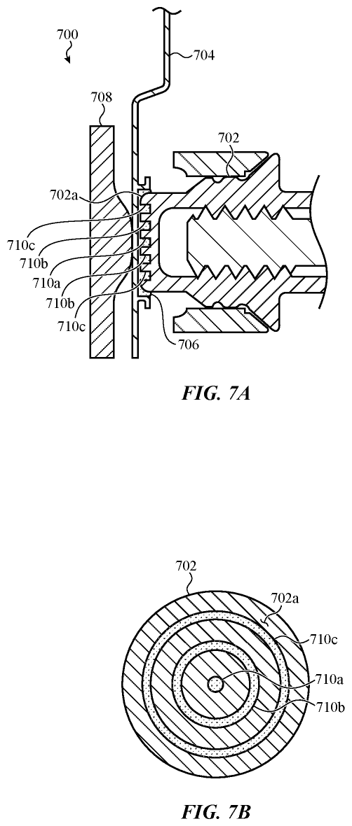

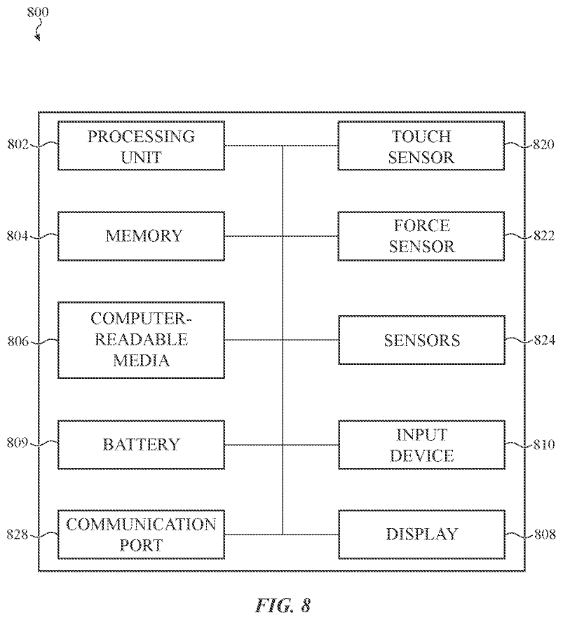

FIGS. 7A-7B shows a variation of an example reservoir configuration 700. In this variation, the crown assembly may include multiple reservoirs arranged in a concentric manner. For example, a shaft 702 may define reservoir 710a, reservoir 710b, and reservoir 710c. Reservoirs 710a-c may be concentric with respect to each other. In some examples, the reservoirs 710a and 710b may define a circular or oval shape, though other geometries such as square, rectangular, hexagonal, and the like, are envisioned. Each reservoir 710a-c may be depressed with respect to an interface surface 702a of the shaft and may be configured to shield portions of the conductive viscous material 706 from dynamic loading to prevent depleting and thus maintain electrical continuity between the shaft 702 and the conductive member 704. By having multiple reservoirs with a concentric arrangement, the volume of conductive viscous material 706 increases compared to a single reservoir, improving the surface area of able to conduct current from the shaft 702 to the conductive member 704. The conductive member 704, in turn, may be configured to actuate dome switch 708 in response to a translational input.

FIG. 8 depicts an example schematic diagram of an electronic device 800. The device 800 of FIG. 8 may correspond to the wearable electronic device 100 shown in FIGS. 1A. To the extent that multiple functionalities, operations, and structures are disclosed as being part of, incorporated into, or performed by the device 800, it should be understood that various examples may omit any or all such described functionalities, operations, and structures. Thus, different examples of the device 800 may have some, none, or all of the various capabilities, apparatuses, physical features, modes, and operating parameters discussed herein.

As shown in FIG. 8, a device 800 includes a processing unit 802 operatively connected to computer memory 804 and/or computer-readable media 806. The processing unit 802 may be operatively connected to the memory 804 and computer-readable media 806 components via an electronic bus or bridge. The processing unit 802 may include one or more computer processors or microcontrollers that are configured to perform operations in response to computer-readable instructions. The processing unit 802 may include the central processing unit (CPU) of the device. Additionally or alternatively, the processing unit 802 may include other processors within the device including application specific integrated chips (ASIC) and other microcontroller devices.

The memory 804 may include a variety of types of non-transitory computer-readable storage media, including, for example, read access memory (RAM), read-only memory (ROM), erasable programmable memory (e.g., EPROM and EEPROM), or flash memory. The memory 804 is configured to store computer-readable instructions, sensor values, and other persistent software elements. Computer-readable media 806 also includes a variety of types of non-transitory computer-readable storage media including, for example, a hard-drive storage device, a solid-state storage device, a portable magnetic storage device, or other similar device. The computer-readable media 806 may also be configured to store computer-readable instructions, sensor values, and other persistent software elements.

In this example, the processing unit 802 is operable to read computer-readable instructions stored on the memory 804 and/or computer-readable media 806. The computer-readable instructions may adapt the processing unit 802 to perform the operations or functions described herein. In particular, the processing unit 802, the memory 804, and/or the computer-readable media 806 may be configured to cooperate with a sensor 824 (e.g., a rotation sensor that senses rotation of a crown component) to control the operation of a device in response to an input applied to a crown of a device (e.g., the crown 112 or any other crown described herein). The computer-readable instructions may be provided as a computer-program product, software application, or the like.

As shown in FIG. 8, the device 800 also includes a display 808. The display 808 may include a liquid-crystal display (LCD), organic light emitting diode (OLED) display, light emitting diode (LED) display, or the like. If the display 808 is an LCD, the display 808 may also include a backlight component that can be controlled to provide variable levels of display brightness. If the display 808 is an OLED or LED type display, the brightness of the display 808 may be controlled by modifying the electrical signals that are provided to display elements. The display 808 may correspond to any of the displays shown or described herein.

The device 800 may also include a battery 809 that is configured to provide electrical power to the components of the device 800. The battery 809 may include one or more power storage cells that are linked together to provide an internal supply of electrical power. The battery 809 may be operatively coupled to power management circuitry that is configured to provide appropriate voltage and power levels for individual components or groups of components within the device 800. The battery 809, via power management circuitry, may be configured to receive power from an external source, such as an AC power outlet. The battery 809 may store received power so that the device 800 may operate without connection to an external power source for an extended period of time, which may range from several hours to several days.

In some examples, the device 800 includes one or more input devices 810. An input device 810 is a device that is configured to receive user input. The one or more input devices 810 may include, for example, a crown input system (e.g., any of the crowns described herein), a push button, a touch-activated button, a keyboard, a keypad, or the like (including any combination of these or other components). In some examples, the input device 810 may provide a dedicated or primary function, including, for example, a power button, volume buttons, home buttons, scroll wheels, and camera buttons.

The device 800 may also include one or more sensors 824. The sensors 824 may detect inputs provided by a user to a crown of the device (e.g., the crown 112 or any other crown described herein). The sensors 824 may include sensing circuitry and other sensing components that facilitate sensing of rotational motion of a crown, as well as sensing circuitry and other sensing components (optionally including a switch) that facilitate sensing of translational and/or axial motion of the crown (or axial force applied to the crown). The sensors 824 may include components such as an optical sensing unit, a tactile or dome switch, or any other suitable components or sensors that may be used to provide the sensing functions described herein. The sensors 824 may also include a biometric sensor, such as a heart rate sensor, electrocardiograph sensor, temperature sensor, or any other sensor that conductively couples to the user and/or to the external environment through a crown input system, as described herein. In cases where the sensors 824 include a biometric sensor, it may include biometric sensing circuitry, as well as portions of a crown that conductively couple a user's body to the biometric sensing circuitry. Biometric sensing circuitry may include components such as processors, capacitors, inductors, transistors, analog-to-digital converters, or the like.

The device 800 may also include a touch sensor 820 that is configured to determine a location of a touch on a touch-sensitive surface of the device 800 (e.g., an input surface defined by the display 108). The touch sensor 820 may use or include capacitive sensors, resistive sensors, surface acoustic wave sensors, piezoelectric sensors, strain gauges, or the like. In some cases, the touch sensor 820 associated with a touch-sensitive surface of the device 800 may include a capacitive array of electrodes or nodes that operate in accordance with a mutual-capacitance or self-capacitance scheme. The touch sensor 820 may be integrated with one or more layers of a display stack (e.g., the display 108) to provide the touch-sensing functionality of a touchscreen. Moreover, the touch sensor 820, or a portion thereof, may be used to sense motion of a user's finger as it slides along a surface of a crown, as described herein.

The device 800 may also include a force sensor 822 that is configured to receive and/or detect force inputs applied to a user input surface of the device 800 (e.g., the display 108). The force sensor 822 may use or include capacitive sensors, resistive sensors, surface acoustic wave sensors, piezoelectric sensors, strain gauges, or the like. In some cases, the force sensor 822 may include or be coupled to capacitive sensing elements that facilitate the detection of changes in relative positions of the components of the force sensor (e.g., deflections caused by a force input). The force sensor 822 may be integrated with one or more layers of a display stack (e.g., the display 108) to provide force-sensing functionality of a touchscreen.

The device 800 may also include a communication port 828 that is configured to transmit and/or receive signals or electrical communication from an external or separate device. The communication port 828 may be configured to couple to an external device via a cable, adaptor, or other type of electrical connector. In some examples, the communication port 828 may be used to couple the device 800 to an accessory, including a dock or case, a stylus or other input device, smart cover, smart stand, keyboard, or other device configured to send and/or receive electrical signals.

It is well understood that the use of personally identifiable information should follow privacy policies and practices that are generally recognized as meeting or exceeding industry or governmental requirements for maintaining the privacy of users. In particular, personally identifiable information data should be managed and handled so as to minimize risks of unintentional or unauthorized access or use, and the nature of authorized use should be clearly indicated to users.

As used herein, the phrase “at least one of” preceding a series of items, with the term “and” or “or” to separate any of the items, modifies the list as a whole, rather than each member of the list. The phrase “at least one of” does not require selection of at least one of each item listed; rather, the phrase allows a meaning that includes at a minimum one of any of the items, and/or at a minimum one of any combination of the items, and/or at a minimum one of each of the items. By way of example, the phrases “at least one of A, B, and C” or “at least one of A, B, or C” each refer to only A, only B, or only C; any combination of A, B, and C; and/or one or more of each of A, B, and C. Similarly, it may be appreciated that an order of elements presented for a conjunctive or disjunctive list provided herein should not be construed as limiting the disclosure to only that order provided.

One may appreciate that although many embodiments are disclosed above, that the operations and steps presented with respect to methods and techniques described herein are meant as exemplary and accordingly are not exhaustive. One may further appreciate that alternate step order or fewer or additional operations may be required or desired for particular embodiments.

Although the disclosure above is described in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the some embodiments of the invention, whether or not such embodiments are described and whether or not such features are presented as being a part of a described embodiment. Thus, the breadth and scope of the present invention should not be limited by any of the above-described exemplary embodiments but is instead defined by the claims herein presented.

What is claimed is:

1. An electronic watch comprising:
a housing comprising a sidewall, the sidewall defining an opening;
a crown defining a user input surface and extending through the opening, the crown configured to receive a least one of a rotational input or a translational input, the crown comprising:
a knob defining the user input surface; and
a shaft extending from the knob, the shaft defining:
a first end coupled to the knob; and
a second end opposite the first end, the second end defining a reservoir; and
a sensing system configured to receive an electrocardiogram (ECG) signal from the user input surface;
a dome switch configured to produce haptic feedback in response to the translational input at the user input surface;
a conductive member conductively coupling the shaft to the sensing system, the conductive member positioned between the dome switch and the second end of the shaft; and
a conductive viscous material between the second end of the shaft and the conductive member, the conductive viscous material filling the reservoir and configured to conductively couple the user input surface to the conductive member.

2. The electronic watch of claim 1, wherein:
the shaft defines a central axis around which the crown rotates; and
the reservoir is centered about the central axis.

3. The electronic watch of claim 1, wherein:
the reservoir is a first reservoir;
the first reservoir is defined by a curved surface depressed with respect to the second end;
the conductive member defines a second reservoir positioned opposite the first reservoir; and
the conductive viscous material fills the second reservoir.

4. The electronic watch of claim 3, wherein the conductive member comprises a stiffening plate at least partially surrounding the second reservoir, the stiffening plate configured to contact the dome switch in response to the translational input.

5. The electronic watch of claim 1, wherein the conductive member is configured to apply a biasing force to the first end of the shaft, the biasing force biasing the crown towards an undepressed position.

6. The electronic watch of claim 1, wherein:
the shaft defines a bottom surface of the reservoir and a wall surface of the reservoir;
the conductive member is positioned at least partially in the reservoir; and
the conductive viscous material is between the bottom surface of the reservoir and the conductive member.

7. The electronic watch of claim 6, wherein the portion of the conductive member contacts the wall surface of the reservoir, thereby defining a seal between the portion of the conductive member and the peripheral wall.

8. An electronic watch comprising:
a processor;
a housing comprising a sidewall, the sidewall defining an opening;
a front cover coupled to the housing;
a display positioned below the front cover;
a flexible circuit element within the housing;
a dome switch positioned within the housing;
a crown positioned along the sidewall of the housing and defining a user input surface configured to receive a translational input, the crown comprising a shaft configured to actuate the dome switch, the shaft extending through the opening and defining an interface surface opposite the user input surface, the interface surface defining a reservoir;
a conductive member configured to conduct an electrical signal from the user input surface to the processor, the conductive member positioned between the dome switch and the crown and conductively coupling the crown to the flexible circuit element; and
a conductive viscous material positioned between the conductive member and the interface surface of the shaft, the conductive viscous material filling the reservoir and conductively coupling the shaft to the conductive member.

9. The electronic watch of claim 8, wherein:
the shaft comprises:
a shaft member having a first end and a second end opposite the first end, the first end of the shaft member coupled to the user input surface of the crown; and
a barrel coupled to the second end of the shaft member, the barrel defining the interface surface and the reservoir; and
the crown is configured to rotate with respect to a central axis of the shaft in response to a rotational input from a user at the user input surface.

10. The electronic watch of claim 8, wherein the conductive viscous material is an electrically conductive grease.

11. The electronic watch of claim 8, wherein the interface surface of the crown is configured to, in response to a rotational input applied to the crown, rotate with respect to the conductive member, thereby applying a shear force to the conductive viscous material.

12. The electronic watch of claim 8, wherein:
the crown, the conductive viscous material, the conductive member, and the flexible circuit element define a conductive path from the user input surface to the flexible circuit element; and
the electrical signal is a physiological signal from a user's skin.

13. The electronic watch of claim 8, wherein:
the conductive member is in contact with the interface surface;
the conductive member is configured to deform in response to the translational input; and
the conductive viscous material is configured to flow during the deformation of the conductive member.

14. The electronic watch of claim 8, wherein:
the reservoir is a reservoir of a plurality of reservoirs having a concentric pattern along the interface surface.

15. A wearable device comprising:
a housing comprising a sidewall, the sidewall defining an opening;
a sensing system configured to receive an electrocardiogram (ECG) signal;
a crown assembly comprising:
a crown defining a user input surface configured to receive the ECG signal, the crown extending through the opening and comprising:
a shaft defining:
a first end portion conductively coupled to the user input surface; and
a second end portion opposite the first end portion; and
a conductive member configured to apply a biasing force against the second end portion of the shaft; and
a conductive viscous material between the conductive member and the second end portion of the shaft, the conductive viscous material configured to conductively couple the conductive member and the shaft, the conductive viscous material conforming to the second end portion of the shaft, wherein:
the crown assembly has a reservoir defined by at least one of the conductive member or the second end portion of the shaft; and
the conductive viscous material fills the reservoir.

16. The wearable device of claim 15, wherein:
the shaft comprises:
a shaft member defining the first end portion of the shaft and extending from the housing; and
a barrel coupled to the shaft member and defining the second end portion of the shaft, the barrel defining an interface surface configured to contact the conductive viscous material;
the barrel defines a peripheral wall that defines the reservoir; and
the conductive member is positioned partially within the peripheral wall trapping the conductive viscous material.

17. The wearable device of claim 15, wherein:
the conductive member defines the reservoir;
the shaft further comprises a barrel defining the second end portion of the shaft and configured to contact the conductive viscous material; and
the end portion of the barrel is positioned within the reservoir, thereby trapping the conductive viscous material between the reservoir and the end portion of the barrel.

18. The wearable device of claim 15, wherein:
the reservoir is a first reservoir;
the first reservoir is defined by the second end portion of the shaft;
the crown assembly further comprises a second reservoir defined by the conductive member; and
the first and second reservoirs are centered with respect to an axis of rotation of the input member.

19. The wearable device of claim 15, wherein:
the wearable device further comprises a sealing member; and
the sealing member is concentric to the reservoir, thereby sealing a portion of the conductive viscous material with respect to the second end portion of the shaft and the conductive member.

20. The wearable device of claim 15, wherein:
the reservoir is centered with respect to a rotation axis of the second end portion of the shaft;
the second end portion defines an interface surface contacting the conductive viscous material; and
the reservoir is defined by a reservoir wall extending orthogonally from the interface surface in a direction opposite the interface surface.
